(19) United States
(12) Patent Application Publication
(10) Pub. No.: US 2019/0177690 A1
(43) Pub. Date: Jun. 13, 2019

(54) METHOD AND SYSTEM FOR CULTURING CORNEAL STEM CELL-LIKE CELL LINE BY INDUCING DIFFERENTIATION OF INDUCED PLURIPOTENT STEM CELL USING PROTEIN LIGAND

(71) Applicant: THE CATHOLIC UNIVERSITY OF KOREA INDUSTRY-ACADEMIC COOPERATION FOUNDATION, Seoul (KR)

(72) Inventors: Choun Ki JOO, Seoul (KR); Jee Won MOK, Seoul (KR); Hee Jung JU, Seoul (KR); Jun Sub CHOI, Gyeonggi-do (KR)

(21) Appl. No.: 16/323,459

(22) PCT Filed: Aug. 4, 2017
(86) PCT No.: PCT/KR2017/008466

§ 371 Date: Feb. 5, 2019

(30) Foreign Application Priority Data
Aug. 10, 2016 (KR) 10-2016-0101954

(51) Int. Cl. C12N 5/079; C12N 5/00

(52) U.S. Cl. CPC C12N 5/0621; C12N 5/0018; C12N 2506/45; C12N 2501/155; C12N 2501/415; C12N 2533/50; C12N 2500/12; C12N 2501/115; C12N 2501/15; C12N 2501/33; C12N 2501/11; C12N 2500/38; C12N 2501/998

(57) ABSTRACT

The present invention relates to a method for culturing corneal stem cell-like cell lines by inducing differentiation of induced pluripotent stem cells (iPSC). The method comprises the following steps:
preparing a basal medium comprising DMEM F12, L-ascorbic acid, sodium selenite, and sodium chloride;
adding additives for culturing induced pluripotent stem cells to the basal medium;
preparing a feeder free culture medium by coating the basal medium with Vitronectin recombinant human protein;
adding induced pluripotent stem cells to the feeder free culture medium and culturing them;
adding additives for creating an environment for stem cell growth to the feeder free culture medium;
adding BMP4 and Wnt3a sequentially to the feeder free culture medium in order to induce differentiation of the induced pluripotent stem cells into corneal stem cell-like cell lines; and
culturing the cells differentiated from the induced pluripotent stem cells in PI culture fluid.

### TECHNICAL FIELD

The present invention relates to a method and a system for culturing corneal stem cell-like cell lines by inducing differentiation of induced pluripotent stem cells using protein ligands.

### BACKGROUND ART

Stem cells are pluripotent cells which are able to differentiate into any cells that make up our body. Theoretically, stem cells can differentiate into any cells. Thus, if we can understand a mechanism of stem cell differentiation and make the stem cell differentiate into a desired cell, it would be possible to restore or regenerate various body organs.

However, since stem cells have the property of proliferating and differentiating by themselves to become cancer cells when they are transplanted into a body, stem cells themselves cannot be used for regenerating or treating tissues.

Further, if the culture period is long and the number of subcultures increases, the capacity of cell line-established induced pluripotent stem cells (iPSC) declines and the possibility of differentiation into an undesired cell or cell death increases, and thus the expected effect is diminished.

Therefore, there is need for a technology for culturing induced pluripotent stem cells while maintaining characteristics of stem cells, and culturing them in a state most suitable for the cells to be differentiated to improve the rate of differentiation of stem cells into desired cells.

### DISCLOSURE

### Technical Problem

The purpose of the present invention is to address the above-described technical problems.

The present inventors came up with a novel method for culturing corneal stem cell-like cell lines by inducing differentiation of induced pluripotent stem cells. The method comprises the following steps:

i) preparing a feeder free culture medium by coating a basal medium with Vitronectin recombinant human protein;

ii) culturing induced pluripotent stem cells in the feeder free culture medium; and

iii) adding BMP4 and Wnt3a sequentially in order to induce differentiation of the induced pluripotent stem cells into corneal stem cell-like cell lines. The present inventors found that the present method provides cells with low immune response upon corneal transplantation and improved differentiation potency of induced pluripotent stem cells into corneal stem cell-like cell lines.

### Technical Solution

Therefore, the method for culturing corneal stem cell-like cell lines by inducing differentiation of induced pluripotent stem cells (iPSC) according to the present invention comprises the following steps:

preparing a basal medium comprising DMEM F12, L-ascorbic acid, sodium selenite, and sodium chloride;

adding additives for culturing induced pluripotent stem cells to the basal medium;

preparing a feeder free culture medium by coating the basal medium with Vitronectin recombinant human protein;

adding induced pluripotent stem cells to the feeder free culture medium and culturing them;

adding additives for creating an environment for stem cell growth to the feeder free culture medium;

adding BMP4 and Wnt3a sequentially to the feeder free culture medium in order to induce differentiation of the induced pluripotent stem cells into corneal epithelial cells; and culturing the cells differentiated from the induced pluripotent stem cells in PI culture fluid.

In one embodiment, the additives for culturing induced pluripotent stem cells may comprise Holo transferrin, bFGF, TGFbeta1, and insulin.

In one embodiment, the additives for creating an environment for stem cell growth may comprise EGF and insulin.

In one embodiment, in the step of adding BMP4 and Wnt3a sequentially, BMP4 may be added and treated for about 2 to 4 days and then Wnt3a may be added, more specifically, BMP4 may be added and treated for about 3 days and then Wnt3a may be added.

In one embodiment, in the step of adding BMP4 and Wnt3a sequentially, Wnt3a may be treated for about 2 to 4 days after being added, more specifically, Wnt3a may be treated for about 3 days after being added.

In one embodiment, the PI culture fluid may comprise Panserin and Iscove's medium at a weight ratio of 2:1 to 1:2, more specifically 1:1.

In one embodiment, the cells differentiated from the induced pluripotent stem cells may be cultured in PI culture fluid for 1 to 3 weeks.

In one embodiment, the method may further comprise a step of subculturing the cells differentiated from the induced pluripotent stem cells after culturing them in PI culture fluid.

In one embodiment, in the step of subculturing, the subculture fluid may comprise Epi medium, FBS, hydrocortisone, insulin, choleratoxin, and EGF.

In one embodiment, the cells differentiated from the induced pluripotent stem cells may be cultured in PI culture fluid for 3 weeks and then subcultured.

The present invention also provides a system for culturing corneal stem cell-like cell lines by inducing differentiation of induced pluripotent stem cells.

The system may comprise

i) a feeder free culture medium which is prepared by adding additives for culturing induced pluripotent stem cells to a basal medium comprising DMEM F12, L-ascorbic acid, sodium selenite, and sodium chloride and coating with Vitronectin recombinant human protein;

ii) induced pluripotent stem cells cultured in the feeder free culture medium;

iii) additives for creating an environment for stem cell growth to be added to the feeder free culture medium;

iv) BMP4 and Wnt3a to be added sequentially to the medium comprising the induced pluripotent stem cells cultured in the feeder free culture medium and the additives for creating an environment for stem cell growth in order to induce differentiation of the induced pluripotent stem cells into corneal stem cell-like cell lines; and

v) PI culture fluid for culturing the cells differentiated from the induced pluripotent stem cells.

In one embodiment, the system may further comprise subculture fluid for subculturing the cells differentiated from the induced pluripotent stem cells after culturing them in PI culture fluid.

### BRIEF DESCRIPTION OF DRAWINGS

FIG. 1 provides a photograph of the induced pluripotent stem cells cultured in a feeder free culture medium of Example 1 over time.

FIG. 2 provides a photograph of the induced pluripotent stem cells cultured in a culture medium comprising feeder of Comparative Example 1 for 7 days.

FIGS. 3 and 4 provide photographs of the results of immunostaining with undifferentiated iPSC markers and protein expression array of the induced pluripotent stem cells cultured in the feeder free culture medium of Example 1.

FIGS. 5 to 8 provide photographs of expression status of markers for Example 1.

FIG. 9 provides photographs of the results of conducting Air Lift for 3 weeks on the cells treated with Wnt3a and cultured for 1 week in PI culture fluid in Example 1.

FIG. 10 provides photographs showing the proliferation rate of cells subcultured in Examples 1 and 2, measured with markers Pax6, CK3, and Hoechst.

FIG. 11 provides a photograph of the result of Air Lift culture of corneal epithelial stem cells cultured in subculture fluid of Example 1.

### BEST MODE

The present invention is further illustrated by the following examples, which are not to be construed to limit the scope of the invention.

### Example 1

To culture induced pluripotent stem cells, a basal medium comprising DMEM F12, L-ascorbic acid, sodium selenite, and sodium chloride was prepared, and adjusted to pH 7.4. As additives for culturing induced pluripotent stem cells, 10 μg/ml of Holo transferrin, 100 ng/mL of bFGF, 1.74 ng/ml of TGFbeta1, and 20 μg/ml of insulin were added to the basal medium.

When stem cells are cultured with a feeder, an immune response may be a problem upon transplantation of differentiated corneal epithelial cells. Thus, to prepare a feeder free culture medium, the basal medium was coated with Vitronectin recombinant human protein and then induced pluripotent stem cells were cultured for 7 days.

Coating with Vitronectin recombinant human protein was conducted as follows. On a 6-well plate culture vessel basis, 60 of Vitronectin recombinant human protein (50 μg/ml) was added to 9 ml of DPBS (phosphate buffered saline without Ca²⁺ and Mg²⁺) for dilution, and 1.5 ml of the mixture was added to each well and then reacted for 12 hours in the cold state (about 4° C.) after being prevented from drying. And then, it was reacted for 1 hour at room temperature and washed with PBS.

To differentiate the cultured induced pluripotent stem cells into corneal epithelial cells, first, 10 ng/ml of EGF and 5 μg/ml of insulin were added as additives for creating an environment for stem cell growth to the feeder free culture medium.

Then, 100 ng/ml of BMP4 was added and treated for 3 days to induce ectoderm progenitors in the early stage, and then 100 ng/ml of Wnt3 a was added and treated for 3 days to differentiate into corneal stem cell-like cell lines.

And then, the cells differentiated from induced pluripotent stem cells were cultured in PI culture fluid which is a mixture of Panserin and Iscove's medium at a weight ratio of 1:1 for 1 to 3 weeks.

Cells cultured in PI culture fluid for 1 to 3 weeks were subcultured. The subculture fluid was prepared by adding 5 wt % of FBS (Fetal Bovine Serum) to Epi medium which is a mixture of DMEM and F12 at a weight ratio of 3:1, and containing 50 ng/ml of hydrocortisone, 5 μg/ml of insulin, 30 ng/ml of choleratoxin, and 10 ng/ml of EGF.

### Example 2

Stem cells were cultured in the same manner as Example 1 except for that the subculture fluid was used with the same composition as the PI culture fluid.

### Comparative Example 1

To culture induced pluripotent stem cells, a basal medium comprising DMEM F12, L-ascorbic acid, sodium selenite, and sodium chloride was prepared, and adjusted to pH 7.4. As additives for culturing induced pluripotent stem cells, 10 μg/ml of Holo transferrin, 100 ng/mL of bFGF, 1.74 ng/ml of TGFbeta1, and 20 μg/ml of insulin were added to the basal medium. 25,000 cells/cm² of MEF (mouse embryonic fibroblast) feeder treated with MMC (mitomycin C) was added as a feeder, and then induced pluripotent stem cells were cultured.

### Experimental Example 1

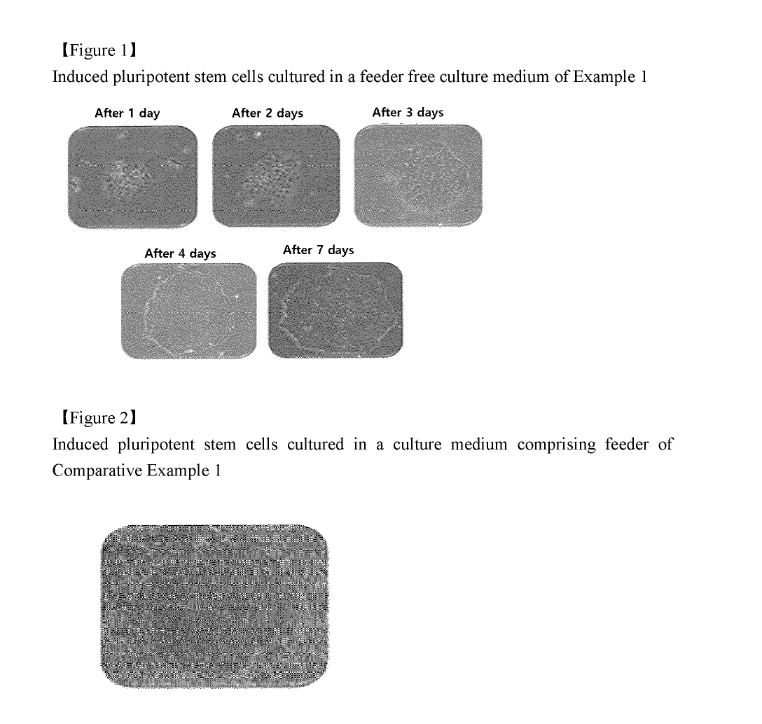

The culture status of the induced pluripotent stem cells cultured in the feeder free culture medium of Example 1 and the induced pluripotent stem cells cultured in the medium comprising feeder of Comparative Example 1 were observed. The photographs of the results are shown in FIGS. 1 and 2.

FIG. 1 provides a photograph of the induced pluripotent stem cells cultured in the feeder free culture medium of Example 1 over time. FIG. 2 provides a photograph of the induced pluripotent stem cells cultured in the culture medium comprising feeder of Comparative Example 1 for 7 days.

Referring to FIGS. 1 and 2, it is shown that the induced pluripotent stem cells cultured in the feeder free culture medium of Example 1 form colonies clearly, compared to the induced pluripotent stem cells cultured in the culture medium comprising feeder of Comparative Example 1. This result shows that the induced pluripotent stem cells of Example 1 have been cultured in a uniform size and shape.

### Experimental Example 2

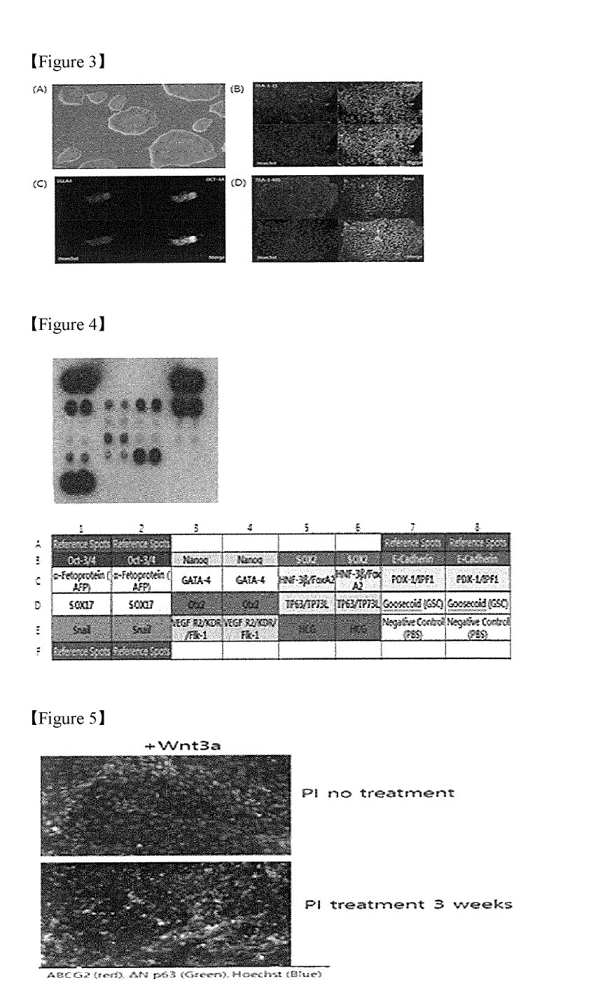

The induced pluripotent stem cells cultured in the feeder free culture medium of Example 1 were immunostained with undifferentiated iPSC markers SOX2, OCT4A, SSEA4, TRA-1-81, and TRA1-60S, and identified by protein expression array. The results are shown in FIGS. 3 and 4.

First, referring to FIG. 3, the presence or absence of expression of TRA-1-81, TRA1-60S, SSEA4, OCT4A which are protein markers indicating that cultured induced pluripotent stem cells (iPSC) have stem cell characteristics were identified.

Then, referring to FIG. 4, proteins were isolated from the cultured induced pluripotent stem cell, and protein array for stem cell markers was conducted to compare protein expression aspects. As a result, the expression of SOX2 and OT3/4 which are markers of stem cells were identified.

### Experimental Example 3

Cells differentiated from the cells differentiated from the induced pluripotent stem cells of Example 1 were analyzed for expression status with corneal stem cell markers (ABCG2, ΔNp63, Pax6, and CK14) and corneal epithelial cell markers (CK3, CK12), before and after cultured in PI culture fluid for 3 weeks, respectively.

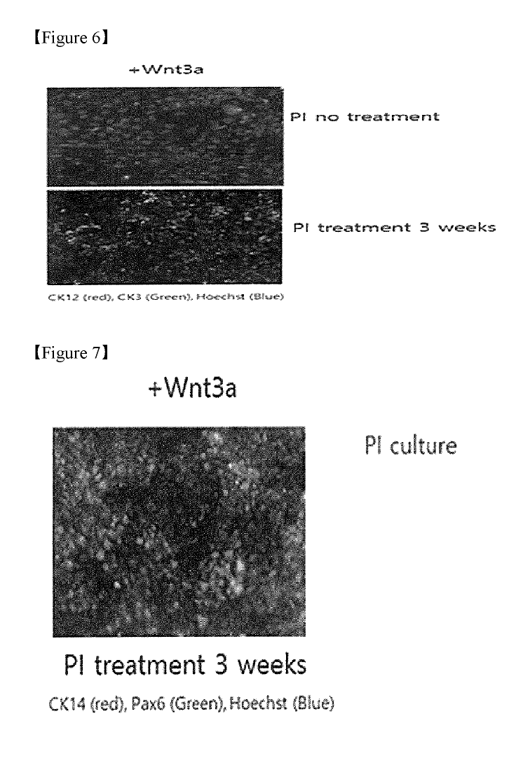

As a result, for Example 1, corneal stem cell markers ABCG2 and ΔNp63 and corneal epithelial cell markers CK3 and CK12 were all observed when the cells are treated with Wnt3a and cultured for 3 weeks. Photographs of expression status of the markers for Example 1 are shown in FIGS. 5 to 8.

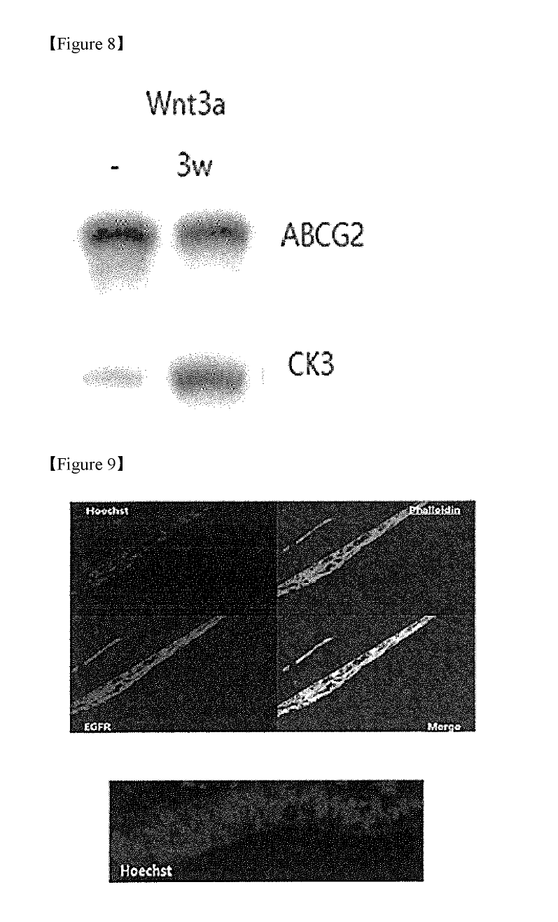

Referring to FIGS. 7 and 8, it is shown that CK14 and Pax6 were expressed in the iPSC treated with Wnt3a and cultured for 3 weeks. Further, it is shown that while the expression of the corneal epithelial stem cell marker ABCG2 decreased in the iPSC treated with Wnt3a, the expression of the corneal epithelial cell differentiation marker CK3 increased.

### Experimental Example 4

Air Lift culture was conducted to identify the differentiation potency of the corneal epithelial stem cells differentiated from the induced pluripotent stem cells of Example 1 into corneal epithelial cells. The result is shown in FIG. 9.

Referring to FIG. 9, it was found that when Air Lift culture was conducted for 3 weeks on the cells of Example 1 treated with Wnt3a and cultured in PI culture fluid for 1 week, about 3 to 4 layers of corneal epithelial cell were formed.

### Experimental Example 5

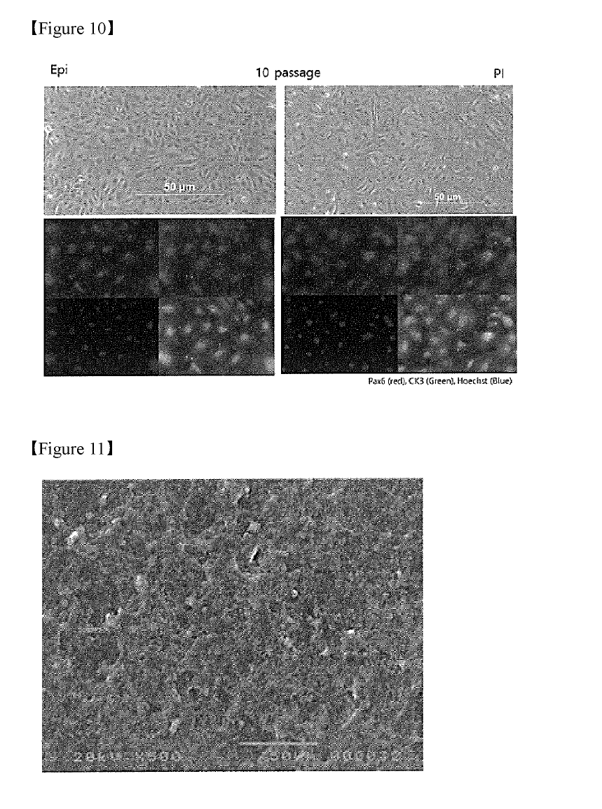

The proliferation rates of the cells subcultured in Example 1 and Example 2 were measured. Markers Pax6, CK3 and Hoechst were used, and the results are shown in FIG. 10 wherein the result of Example 1 using the subculture fluid comprising Epi medium is shown on the left and the result of Example 2 using PI culture fluid as a subculture fluid is shown on the right.

Referring to FIG. 10, it is found that Example 1 using the subculture fluid comprising Epi medium shows much higher density.

Further, the cells were cultured in PI culture fluid for 1 week, 2 weeks, and 3 weeks before subculturing, and then were subcultured, respectively. The cells subcultured after culturing in PI culture fluid for 3 weeks maintained the expression of corneal epithelial cell markers and corneal epithelial stem cell markers constantly while maintaining the shape of corneal epithelial cells best. Through this, the corneal stem cell-like cell lines having at least 70% of differentiation rate could be obtained.

### Experimental Example 6

As a result of Air Lift culture of the corneal epithelial stem cells cultured in the subculture fluid comprising Epi medium, it was observed that multi layers of cells were formed by electron microscope. The result is shown in FIG. 11 which shows that multi layers of cells were formed.

While the invention has been described with respect to the above specific examples, it should be recognized that various modifications and changes may be made to the invention which also fall within the scope of the invention by those skilled in the art.

### INDUSTRIAL APPLICABILITY

As described above, the present method and system for culturing corneal stem cell-like cell lines by inducing differentiation of induced pluripotent stem cells can provide corneal epithelial cells with low immune response upon corneal transplantation.

The present method and system can improve the differentiation potency of induced pluripotent stem cells into corneal stem cell-like cell lines.

The corneal stem cell-like cell lines provided by the present method and system can be used in a study of differentiation of corneal epithelial cells and a study of corneal epithelial transplantation for treatment of corneal diseases.

1. A method for culturing corneal stem cell-like cell lines by inducing differentiation of induced pluripotent stem cells, comprising the following steps:
i) adding BMP4 and Wnt3a sequentially to a feeder free culture medium in order to induce differentiation of induced pluripotent stem cells into corneal stem cell-like cell lines; and
ii) culturing the cells differentiated from the induced pluripotent stem cells.

2. The method according to claim 1 further comprising the following steps before inducing differentiation into corneal epithelial cells:
i) preparing a feeder free culture medium;
ii) adding induced pluripotent stem cells to the feeder free culture medium and culturing them; and
iii) adding additives for creating an environment for stem cell growth to the feeder free culture medium.

3. The method according to claim 2, wherein the step of preparing a feeder free culture medium comprises the following steps:
i) preparing a basal medium comprising DMEM F12, L-ascorbic acid, sodium selenite, and sodium chloride;
ii) adding additives for culturing induced pluripotent stem cells to the basal medium; and
iii) preparing a feeder free culture medium by coating the basal medium with Vitronectin recombinant human protein.

4. The method according to claim 1, wherein the cells differentiated from the induced pluripotent stem cells are cultured in PI culture fluid.

5. The method according to claim 3, wherein the additives for culturing induced pluripotent stem cells comprise Holo transferrin, bFGF, TGFbeta1, and insulin.

6. The method according to claim 2, wherein the additives for creating an environment for stem cell growth comprise EGF and insulin.

7. The method according to claim 1, wherein in the step of adding BMP4 and Wnt3a sequentially, BMP4 is added and treated for 2 to 4 days and then Wnt3a is added.

8. The method according to claim 7, wherein in the step of adding BMP4 and Wnt3a sequentially, Wnt3a is treated for 2 to 4 days after being added.

9. The method according to claim 4, wherein the PI culture fluid comprises Panserin and Iscove's medium at a weight ratio of 2:1 to 1:2.

10. The method according to claim 4, wherein the cells differentiated from the induced pluripotent stem cells are cultured in PI culture fluid for 1 to 3 weeks.

11. The method according to claim 10, further comprising a step of subculturing the cells differentiated from the induced pluripotent stem cells after culturing them in PI culture fluid.

12. The method according to claim 11, wherein in the step of subculturing, the subculture fluid comprises Epi medium, FBS, hydrocortisone, insulin, choleratoxin, and EGF.

13. The method according to claim 11, wherein the cells differentiated from the induced pluripotent stem cells are cultured in PI culture fluid for 3 weeks and then subcultured.

14. A system for culturing corneal stem cell-like cell lines by inducing differentiation of induced pluripotent stem cells comprising:
i) a feeder free culture medium which is prepared by adding additives for culturing induced pluripotent stem cells to a basal medium comprising DMEM F12, L-ascorbic acid, sodium selenite, and sodium chloride and coating with Vitronectin recombinant human protein;
ii) induced pluripotent stem cells cultured in the feeder free culture medium;
iii) additives for creating an environment for stem cell growth to be added to the feeder free culture medium;
iv) BMP4 and Wnt3a to be added sequentially to the medium comprising the induced pluripotent stem cells cultured in the feeder free culture medium and the additives for creating an environment for stem cell growth in order to induce differentiation of the induced pluripotent stem cells into corneal epithelial cells; and
v) PI culture fluid for culturing the cells differentiated from the induced pluripotent stem cells.

15. The system according to claim 14, further comprising a subculture fluid for subculturing the cells differentiated from the induced pluripotent stem cells after culturing them in PI culture fluid.
